(19) United States
(12) Patent Application Publication
(10) Pub. No.: US 2022/0157438 A1
(43) Pub. Date: May 19, 2022

(54) UNDERPAYMENT IDENTIFICATION TOOL AND REVENUE RECOVERY PROCESS

(71) Applicant: Acuet RCM, LLC, Chicago, IL (US)

(72) Inventor: Michael A. ORSENO, Chicago, IL (US)

(73) Assignee: Acuet RCM, LLC, Chicago, IL (US)

(21) Appl. No.: 16/950,884

(22) Filed: Nov. 17, 2020

(51) Int. Cl. G16H 40/20; G06Q 10/10; G16H 70/20; G06Q 40/08; G06Q 40/00; G06Q 30/04

(52) U.S. Cl. CPC G16H 40/20; G06Q 10/10; G16H 70/20; H04L 67/12; G06Q 40/12; G06Q 30/04; G06Q 40/08

(57) ABSTRACT

A computer-implemented underpayment assessment tool, and accompanying process, for comprehensive, full-spectrum examination and analysis of healthcare provider revenue cycle billing data to identify claim payment variances, identify variances as recoverable or non-recoverable, and capture recoverable underpayments.

### FIELD OF THE INVENTION

The present invention pertains to a computer-implemented claim underpayment identification tool and a procedure to utilize claim underpayment identification in the revenue recovery process. More specifically, the present invention pertains to healthcare billing claims, identification of underpayments on billed claims, and recovery of underpayments from health insurance payers and/or healthcare patients.

### INVENTION BACKGROUND

Revenue cycle management is the process used by healthcare providers to track the revenue from patients, from the initial appointment with a provider or admission into a healthcare system to the final payment of invoices from the provider or healthcare system. Medical claim billing is getting increasingly more complicated and errors in revenue cycle management can lead to the healthcare provider receiving delayed payments or no payment at all from insurance company payors. According to Healthcare Financial Management Association statistics, payor underpayments typically total and estimated 7-11% of healthcare provider net revenue.

Healthcare provider claim billing and insurance company claim payment involves a complex process of: (1) coding healthcare events provided to patients, (2) submitting claims to the payers for payment, and, when applicable, (3) insurance company payers processing the claims and paying the provider in accordance to provider/payer contract constraints. The complexities in the system arise from the many codes, variables, and contract parameters associated with each claim submission. In coding healthcare events, healthcare providers (either directly or through outsourced, third party billing services) assign a value from the approximately 10000 Current Procedural Terminology code (CPT codes) specified by the American Medical Association annually. In addition to CPT codes, the healthcare provider specifies billing modifier codes that may affect payer claim payment calculations.

When the patient has insurance covering the healthcare event, the insurance company receives the claim from the healthcare provider and processes the claim according to payment parameters from the provider/payer contract. These parameters include claim payment methodology (e.g., % of charges or set amount per fees schedule), multiple procedural discounts, case rates, and implant reimbursement. The outcome of the claim adjudication is a determination whether the claim can be paid and the amount of the payment to the provider.

With the number of inputs, variables, and computations on both the provider and payer side, errors are a certainty and generally favor underpayment to the provider. Current methodologies for carrying out audits of healthcare claim payments from payers is highly labor intensive and timely. Consequently, only a small sample of a healthcare provider's claim portfolio can be examined and analyzed for errors and underpayments. The result is difficulty in accurately extrapolating potential monetary losses across all provider encounters. The small sample size is typically useful in pointing out the need for future changes to the provider billing practices but provides limited useful information for providers to pursue and capture the underpaid amounts on claims. If providers are collecting from payers at an acceptable rate of return, potential losses may be considered just the cost of doing business.

The present invention is directed to a system and methodology addressing the shortcomings and limitations of current audit procedures. The inventive system and methodology enable the audit to analyze the entirety of all claims selected according to provider criteria to determine all variances in expected claim payments and actual claim payments from all payer sources. Once variances are identified, they can be characterized as permissible/acceptable underpayment, or flagged for lost revenue recovery action.

### SUMMARY OF INVENTION

A computer-implemented tool for examining healthcare provider revenue cycle data and identifying healthcare claim underpayments by healthcare claim payers, the tool comprises: (a) a storage module to electronically store a first data set comprising the provider revenue cycle data and a second data set comprising at least a first set of healthcare claim payer claim payment constraints; (b) a processing module to compare selected data elements from the first and second data sets and generate a third data set comprising underpayment data, the third data set electronically stored in the storage module of the tool; and (c) an output module configured to prepare a claim underpayment report from the third data set and display the claim underpayment report. The computer-implemented tool further comprises a communications module enabling electronic communication between the tool and a provider management information system enabling the tool to communicate with the provider management information system to electronically obtain the provider revenue cycle data. In another embodiment, the computer-implemented tool comprises a data input module enabling the system to obtain the provider revenue cycle data by direct data entry or direct file upload from an electronic storage medium. The provider revenue cycle data comprises a plurality of data elements including: at least a first CPT code, optionally at least a first billing modifier associated with the at least a first CPT code, a first monetary amount representing a claim amount billed and associated with the at least a first CPT code, a second monetary amount representing a claim amount collected by the healthcare provider.

The computer-implemented tool may also comprise a data input module enabling the tool to obtain the at least a first set of healthcare claim payer claim payment constraints by direct data entry or direct file upload from an electronic storage medium. In another embodiment the computer-implemented tool comprises a communications module enabling electronic communication between the tool and a payer management information system and wherein the tool communicates with the payer management information system to electronically obtain the at least a first set of healthcare claim payer claim payment constraints. The first set of healthcare claim payer claim payment constraints comprises a plurality of data elements including: healthcare claim reimbursement methodology, payer procedural discounts, case rates, and implant reimbursements. The second data set may also comprise claim payment constraints from a plurality of healthcare claim payers.

The first data set comprises all the provider revenue cycle data or essentially all the provider revenue cycle data from a specified timeframe and where, in one embodiment, the specified timeframe is selected from a range between 30 days and 180 days.

### BRIEF DESCRIPTION OF FIGURES

FIG. 1 is a schematic diagram of the hardware components of the present inventive underpayment assessment tool;

FIG. 2 shows a flow diagram of the processing logic of the present inventive underpayment assessment tool;

FIGS. 3-10 show graphic representations of user interface screen for the present inventive underpayment assessment tool; and

FIG. 11 and FIG. 12 show flow diagrams of underpayment assessment analysis using the present inventive underpayment assessment tool.

### DETAILED DESCRIPTION OF FIGURES

The computer-implemented system, and accompanying process, disclosed and described herein is a tool for comprehensive, full-spectrum examination and analysis of healthcare provider revenue cycle billing data to identify claim payment variances, identify variances as recoverable or non-recoverable, and capture recoverable underpayments. Existing methodologies consider a small sample size identifying only a potentially statistically insignificant number of variances useful to highlight the need for changes to billing practices. The comprehensive, full-spectrum examination and analysis provided by the inventive system and process provides the accuracy and certainty across the entire provider data set. This precision is required for providers to pursue and capture underpayment amounts from payers, either insurance company or individual patients.

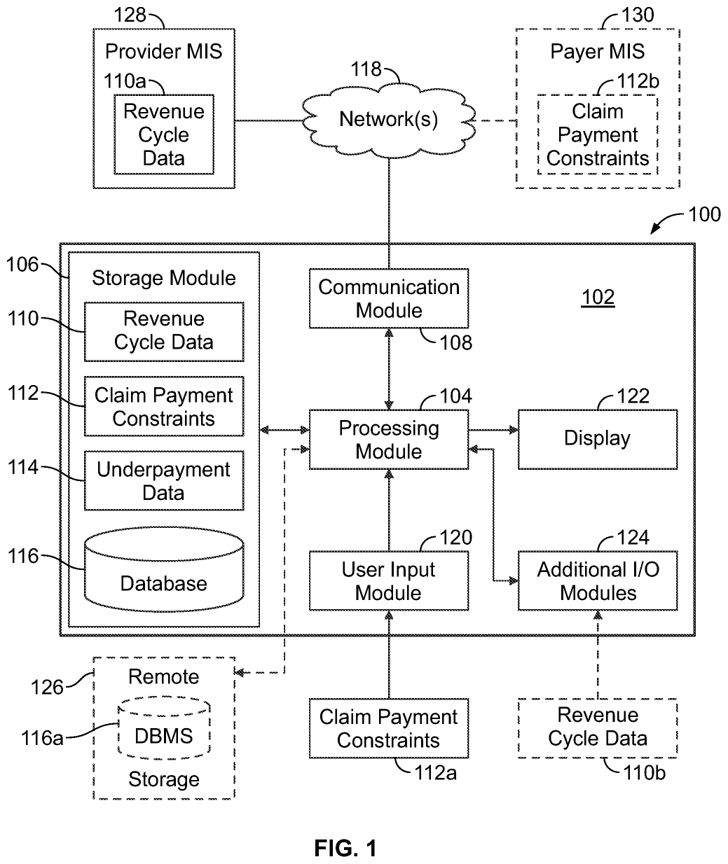

Referring now to FIG. 1, the hardware components of an exemplary underpayment identification tool 100 are shown. The underpayment identification tool 100 may comprise a suitably programmed computing unit 102 in the form of a server computer, desktop computer, laptop computer, handheld computer, or the like. The computing unit 102 includes a processing module 104, storage module 106, and communication module 108, wherein the processing module 104 is operatively connected to both the storage module 106 and communication module 108. The storage module 106 stores revenue cycle data 110 from a provider, claim payment constraints 112 from a payer, underpayment report data 114, and database 116. The processing module 104 can include one or more processing devices such as a microprocessor, microcontroller, digital signal processor or combinations thereof capable of executing instructions associated with the underpayment identification tool 100 and its various modules. Likewise, the storage module 106 can include one or more storage devices such as volatile or non-volatile memory including random access memory (RAM), read-only memory (ROM), electrically-erasable programmable read-only memory (EEPROM), and/or other suitable storage devices.

The communication module 108 can be a network interface including hardware and/or software allowing processing module 104 to communicate with other devices via a wired and/or wireless network(s) 118, as known in the art. The network(s) 118 comprise any suitable communication network such as the World Wide Web, the Internet, Ethernet, VW-Fi, and/or IEEE 802.11 for example. The communication module 108 can be any suitable network interface capable of interacting with the network(s) such as, for example, an Ethernet interface, USB interface, and/or a wireless interface.

The underpayment identification tool 100 can include one or more user input module(s) 120, a display 122, and/or additional input/output module(s) 124, each in communication with the processing module 104. The user input module(s) 110 can be any known mechanism for providing user input to the processing module 104. For example, the user input module(s) 120 can include a keyboard, a mouse, a touch screen, stylus and/or any other suitable means to provide input data to the processing module 104. The display 122 can include any conventional display mechanism such as a cathode ray tube (CRT), a flat panel display, a liquid crystal (LCD) display, a light emitting diode (LED) display, plasma display, and/or any other suitable display mechanism known to those having ordinary skill in the art. Techniques for providing display data from the processing module 104 to the display 122 are well known in the art. The additional (optional, as illustrated by the dashed lines) input/output module(s) 124 can include various media drives (such as magnetic disc or optical disc drives), a microphone or any other source of input data or selection indications, as well as other devices capable of providing information to a user of the underpayment identification tool 100, such as speakers, LEDs, tactile outputs, and other suitable devices.

FIG. 1 shows the revenue cycle data 110 in the storage module 106 originating from a management information system (MIS) 128 of the provider. The revenue cycle data 110a stored in the provider MIS 128 is communicated to the tool 100 via the network 118 and communication module 108. The processing module 104 directs the incoming revenue cycle data 110a to the storage module 106 where it is electronically stored (as 110) for further computation and use by the underpayment identification tool 100. Alternatively, revenue cycle data 110b (shown in the dashed line in FIG. 1) can be entered into the computing unit 102 through the additional input/output modules 124 (e.g., keyed in directly through a keyboard or uploaded from a storage medium including, but not limited to CD/ROM, USB drive, Solid State Drive, and the like).

FIG. 1 shows claim payment constraints 112a in the storage module 106 being entered into the tool 100 through the user input module 120. This input can occur by directly keying the claim payment constraints 112a into the system through use of a keyboard or uploading the claim payment constraints 112a from suitable storage media. As with the revenue cycle data 110, the processing module 104 directs the incoming claim payment constraints 112a to the storage module 106 where the claim payment constraints are electronically stored (as 112) for further computation and use by the underpayment identification tool 100. Alternatively, claim payment constraints 112b may be stored at a management information system (MIS) 130 of the payer (shown in dashed lines in FIG. 1). The claim payment constraints 112b stored in the payer MIS 130 are communicated to the underpayment assessment tool 100 through the communications module 108. The processing module 104 directs the claim payment constraints 112b to the storage module 106 where it is stored (as 112) for further computation and use by the tool 100.

Using the communication module 108, the inventive underpayment identification tool 100 can operate in a remote manner, for example, as in the case of a Web application service. Additionally, in some embodiments, the database 116a is stored in a remote storage module 126, which is in communication with the underpayment identification tool 100 via the network(s) 118. The remote storage module 126 can be any suitable database server with appropriate software such as a database management system (DBMS) for example.

The processing module 104 and/or user of the tool 100 analyzes the revenue cycle data 110 to confirm it is in the proper format for further processing 204. If “no”, the revenue cycle data is re-formatted to the proper format 206, preferably at the provider MIS 128 or manually by the tool user. Once the revenue cycle data 110 is in the proper format it is saved as a first data set in the storage module 106. Insurance company payer contracts are then obtained 208 and analyzed by the tool user 210. Properly formatted claim payment constraints 112 are uploaded to the system 212 and saved in the storage module 106 as a second data set. The claim payment constraints 112 generally include: the payer reimbursement methodology (“% of charges” or “fee schedule/set amount per CPT code”), payer multiple procedural discounts (e.g., multiple CPT codes billed during a single healthcare event), case rates (carve outs/exceptions for grouping related CPT codes), and/or implant reimbursement.

The underpayment assessment tool 100 analyzes the first data set (revenue cycle data 110) in view of the second data set (claim payment constraints 112) and prepares a third data set comprising underpayment data 114 saved in the storage module 106. The underpayment data 114 is displayed on the tool display 122, and the data is reviewed for validation errors 214. The validation can be performed manually by the tool user and/or automatically by the tool 100. When validation errors are located 216, the provider is contacted for correction to the data or provision of clarifying information and the processing steps re-run. When there are no validation errors, the underpayment report 218 is analyzed to determine if a variance between the payment the provider is expecting the actual payment by the payer is recoverable or non-recoverable/permissible underpayment. Recoverable variances can include provider billing errors, payer calculation errors (typically insurance company payer errors), challengeable payer claim denials (e.g., improper medical necessity-based denials or lacking preauthorization denials), and/or secondary coverage or benefits calculations 220. Non-recoverable variances can include patient bills sent to an outside collection agency, patient bankruptcy or insolvency, a logged CPT code is not covered by the payer, or the variance is less than a preset amount (e.g., $5.00). With the analyzed underpayment report the provider can determine the need and best strategy to capture lost revenue 222.

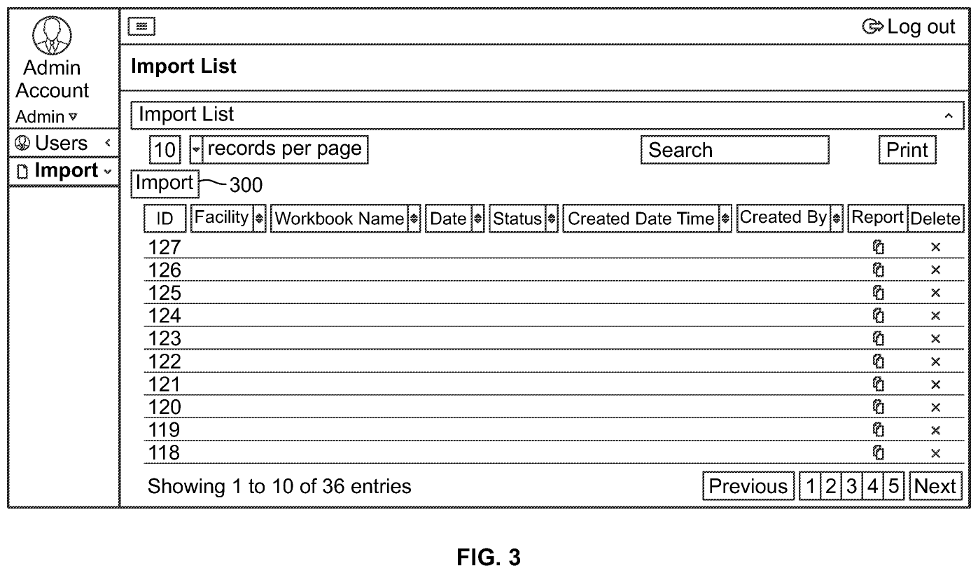

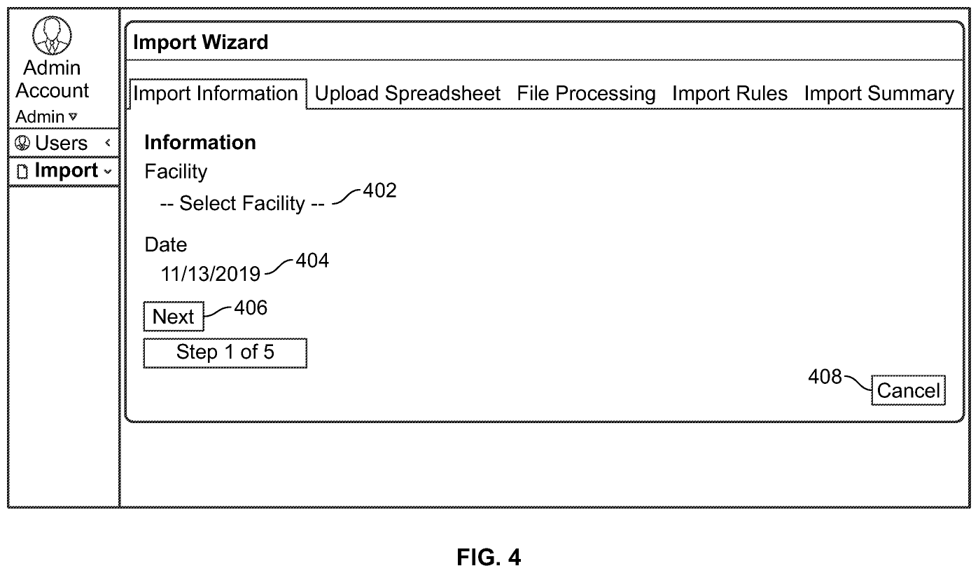

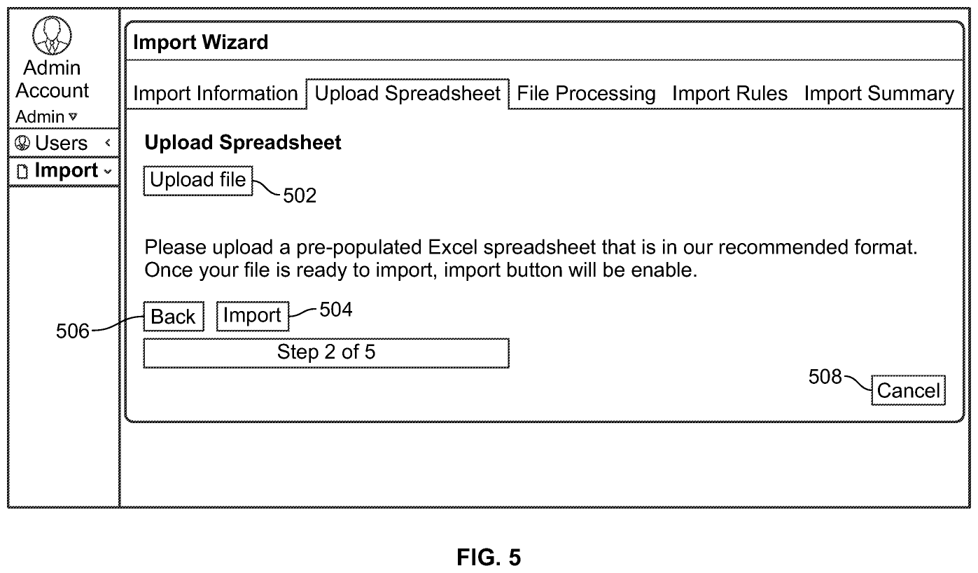

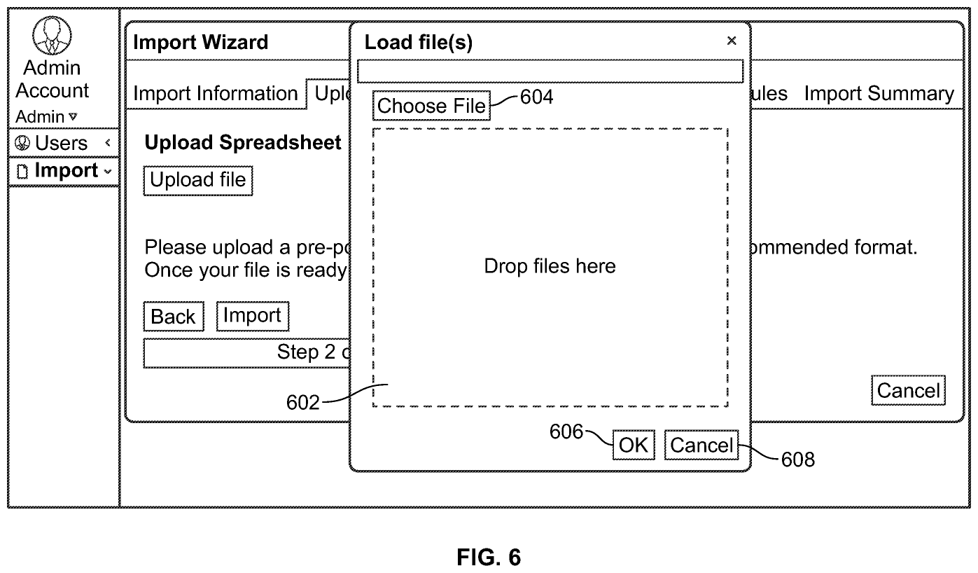

FIG. 3 through FIG. 10 show various screens of the graphical user interface of the tool 100. In FIG. 3, the tool user initiates the process inputting data (both revenue cycle data and claim payment constraints) into the tool 100 by clicking the “Import” button 300. A summary of uploaded data and reports is also viewable on this screen. In FIG. 4, the user initiates an upload procedure with a specific provider by selecting the provider from the “—Select Facility—” drop down menu 402 and selecting a date or date range from the “—Date—” field 404. The “Next” function 406 brings the user to the “Upload Spreadsheet” screen of FIG. 5. The upload process can be cancelled with the “Cancel” function 408. FIG. 5, spreadsheet files are uploaded to the system. “Upload file” 502 moves the user to a new screen (FIG. 6) select and upload a file. As seen in FIG. 6, a spreadsheet file can be “dragged and dropped” into the system 602 or chosen through file server tree 604. Once a file is selected, the user can complete the upload with the “OK” button 606 or cancel the process with the “Cancel” button 608. Returning to FIG. 5, the import can be completed using the “Import” button 504. Alternatively, the user can return to the “Import Information” screen (FIG. 4) with the “Back” function 506 or cancel the operation altogether with the “Cancel” function 508.

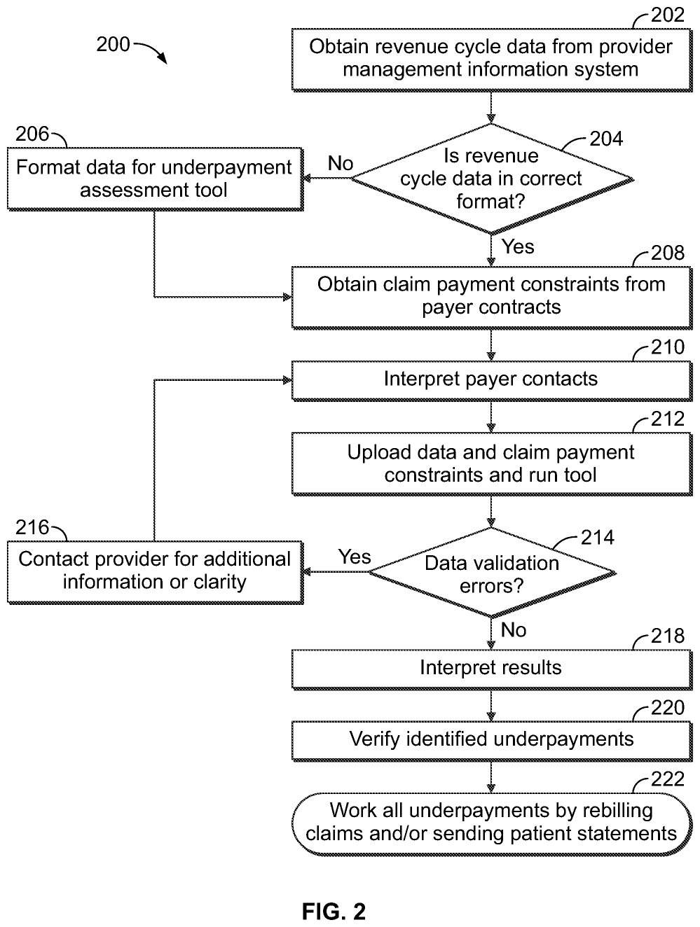

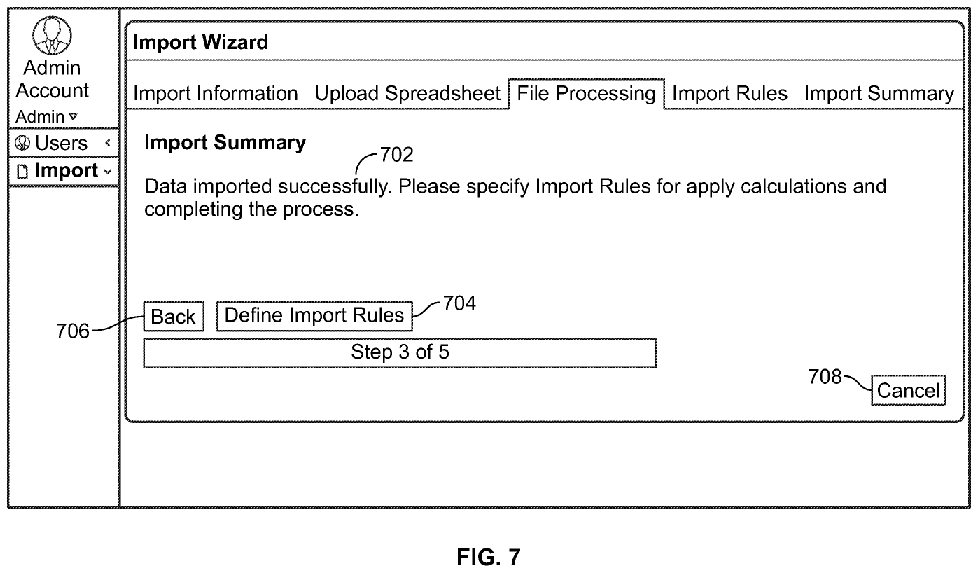

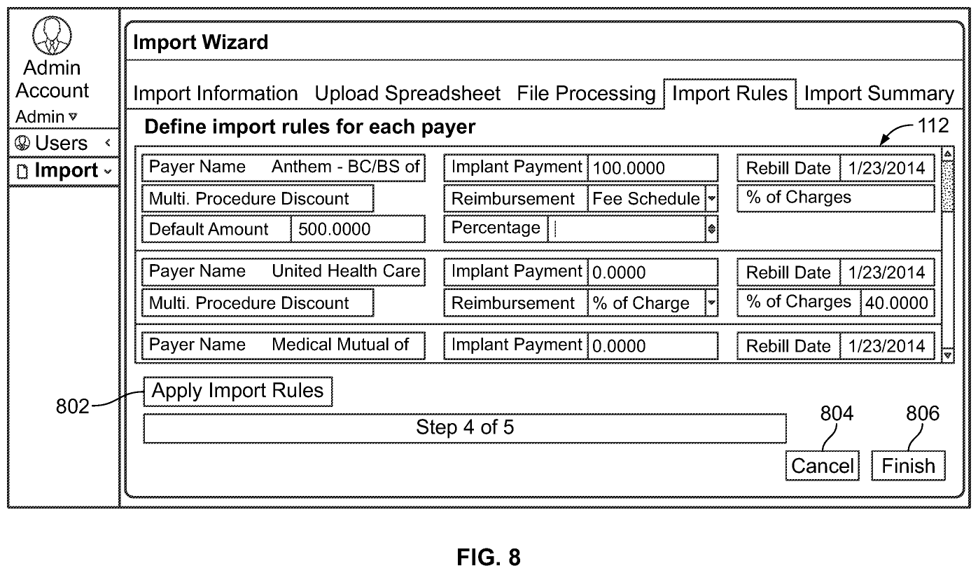

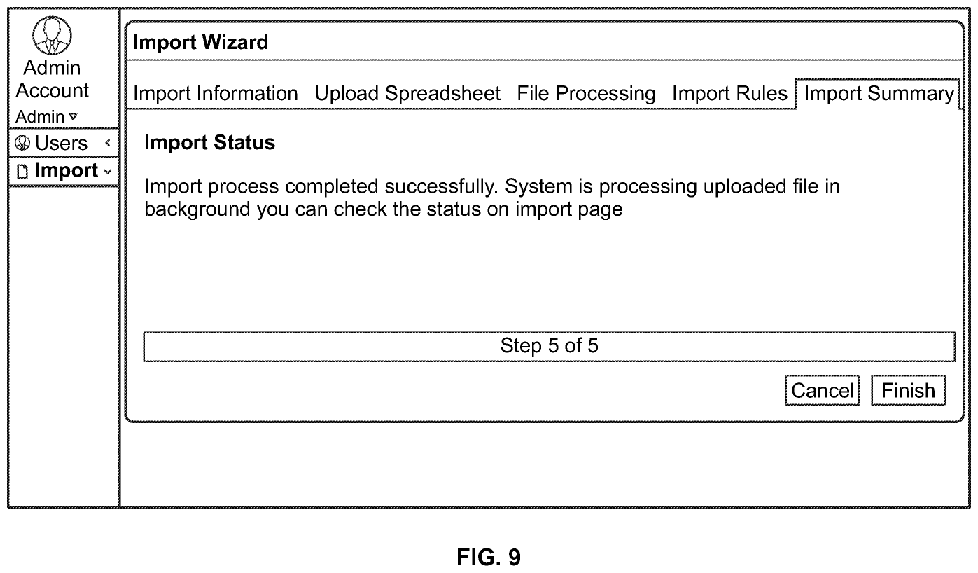

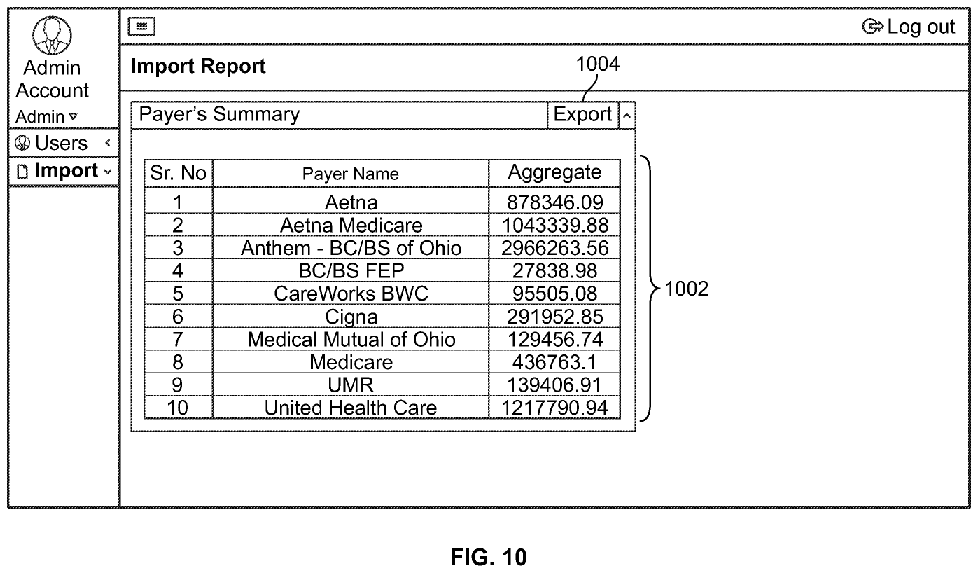

Turning to FIG. 7, after executing the “Import” function (FIG. 5) a “Data imported successfully” message 702 will be displayed if/when data is successfully imported. The user can initiate the procedure to enter and process the payer claim payment constraints with “Define Import Rules” function 704. At this screen the user can also return to the Upload Spreadsheet screen (FIG. 5) with the “Back” function 706 or cancel the entire operation with the “Cancel” function 708. Referring to FIG. 8, claim payment constraints 112 are keyed directly into the system or, alternatively, properly formatted data is uploaded to the system to populate the claim payment constraint fields. Once all claim payment constraints are entered and reviewed by the user, the “Apply Import Rules” function 802 completes the uploading and data entry process. The “Cancel” function 804 can cancel the entire operation and the “Finish” function 806 initiates processing of the revenue cycle data against the claim payment constraints to generate the underpayment data. FIG. 9 shows a ‘placeholder’ screen notifying the user the data processing is in place. FIG. 10 provides a summary of the claim payments 1002 by payers to the specified provider (from FIG. 2). The “Export” function 1004 sends the underpayment data, for example to a suitable database or spreadsheet software, for further review and processing.

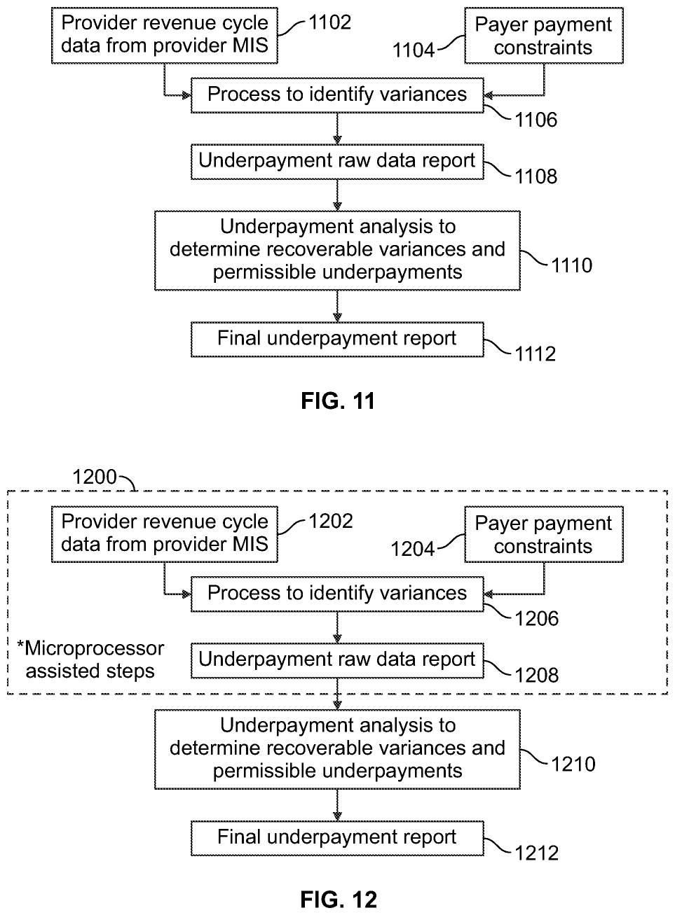

FIG. 11 and FIG. 12 show a method for generating an underpayment report using the inventive underpayment assessment tool described herein. In the first steps 1102/1202, 1104/1204, provider revenue cycle data (first data set) and payer claim payment constraints (second data set) are collected and processed 1106/1206 to identify variances between the payments expected by the provider and payments made by the payer. An underpayment raw data report (third data set) is generated 1108/1208 for further review and further processing. The underpayment raw data report is analyzed 1110/1210 to identify recoverable variances and permissible variances. Recoverable variances are those where errors in data entry and/or processing result in the need for further review and re-billing to avoid lost revenues. Permissible variances may not meet a predetermined, minimum threshold for review and re-billing. A final underpayment report is generated 1112/1212 after applying rules to identify recoverable and permissible variances. FIG. 12 further shows the steps of the method performed by the inventive underpayment assessment tool 1200 in one embodiment of the invention.

The appended figures provide graphic representations of the visual interface of the underpayment identification tool of the present invention and flow charts representing the logic of the inventive underpayment identification tool and revenue recovery process.

Those skilled in the art will appreciate that the invention described herein is susceptible to variations and modifications other than those specifically described. It is to be understood that the invention includes all such variations and modifications that fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A computer-implemented tool for examining healthcare provider revenue cycle data and identifying healthcare claim underpayments by healthcare claim payers, the tool comprising:
a. a storage module to electronically store a first data set comprising the provider revenue cycle data and a second data set comprising at least a first set of healthcare claim payer claim payment constraints;
b. a processing module to compare selected data elements from the first and second data sets and generate a third data set comprising underpayment data, the third data set electronically stored in the storage module of the tool; and
c. an output module configured to prepare a claim underpayment report from the third data set and display the claim underpayment report.

2. The computer-implemented tool of claim 1 further comprising a communications module enabling electronic communication between the tool and a provider management information system.

3. The computer-implemented tool of claim 2 wherein the tool communicates with the provider management information system to electronically obtain the provider revenue cycle data.

4. The computer-implemented tool of claim 1 further comprising a data input module enabling the system to obtain the provider revenue cycle data by direct data entry or direct file upload from an electronic storage medium.

5. The computer-implemented tool of claim 1 wherein the provider revenue cycle data comprises a plurality of data elements including: at least a first CPT code, optionally at least a first billing modifier associated with the at least a first CPT code, a first monetary amount representing a claim amount billed and associated with the at least a first CPT code, a second monetary amount representing a claim amount collected by the healthcare provider.

6. The computer-implemented tool of claim 1 further comprising a data input module enabling the tool to obtain the at least a first set of healthcare claim payer claim payment constraints by direct data entry or direct file upload from an electronic storage medium.

7. The computer-implemented tool of claim 1 further comprising a communications module enabling electronic communication between the tool and a payer management information system and wherein the tool communicates with the payer management information system to electronically obtain the at least a first set of healthcare claim payer claim payment constraints.

8. The computer-implemented tool of claim 1 wherein the first set of healthcare claim payer claim payment constraints comprises a plurality of data elements including: healthcare claim reimbursement methodology, payer procedural discounts, case rates, and implant reimbursements.

9. The computer-implemented tool of claim 1 wherein the second data set comprises claim payment constraints from a plurality of healthcare claim payers.

10. The computer-implemented tool of claim 1 wherein the first data set comprises all the provider revenue cycle data or essentially all the provider revenue cycle data from a specified timeframe.

11. The computer-implemented tool of claim 10 wherein the specified timeframe is selected from a range between 30 days and 180 days.

12. The computer-implemented tool of claim 1 further comprising manually reviewing the claim underpayment report to determine permissible underpayments and recoverable underpayments.

13. A computer-aided healthcare billing revenue recovery process for identifying underpayments and recovering revenues, the process comprising:
a. obtaining healthcare provider revenue cycle data over a predetermined timeframe from a management information system of the provider, uploading and electronically storing the revenue cycle data in a storage module of a computer system;
b. determining if the revenue cycle data is in a proper format for processing by a processing module of the computer system;
c. obtaining health insurance payer contracts from a payer;
d. interpreting payer contracts, uploading and electronically storing data and payer claim payment constraints in the storage module of the computer system;
e. identifying validation errors in the revenue cycle data or claim payment constraints;
f. calculating payment variance data with the processing module of the computer system;
g. interpreting underpayments and verifying collectable and non-collectable variances;
h. work all underpayments by rebilling claims, following-up with insurance companies, and/or sending patient statements.
